(19) United States
(12) Patent Application Publication
(10) Pub. No.: US 2017/0307590 A1
(43) Pub. Date: Oct. 26, 2017

(54) INHIBITORS OF MALARIAL AND HUMAN GLUCOSE TRANSPORTERS AND METHODS FOR IDENTIFYING THE INHIBITORS

(71) Applicant: Washington University in St. Louis, St. Louis, MO (US)

(72) Inventors: Paul Hruz, St. Louis, MO (US); Thomas E. Kraft, St. Louis, MO (US); Brian DeBosch, St. Louis, MO (US)

(21) Appl. No.: 15/492,550

(22) Filed: Apr. 20, 2017

Related U.S. Application Data
(60) Provisional application No. 62/325,148, filed on Apr. 20, 2016.

(51) Int. Cl. G01N 33/50; A61K 31/4745; G01N 33/68

(52) U.S. Cl. CPC G01N 33/5035; G01N 33/6872; A61K 31/4745; G01N 2333/445

(57) ABSTRACT

Inhibitors of glucose transporter Plasmodium falciparum hexose transporter (PfHT) are provided herein. Further, a cell-based, high-throughput assay that directly measures the ability of a compound to inhibit glucose transport by PfHT is provided.

### CROSS-REFERENCE TO RELATED APPLICATIONS

This application claims priority to U.S. Provisional Patent Application Ser. No. 62/325,148, filed on Apr. 20, 2016, the disclosure of which is hereby incorporated by reference in its entirety.

### STATEMENT IN SUPPORT FOR FILING A SEQUENCE LISTING

A paper copy of the Sequence Listing and a computer readable form of the Sequence Listing containing the file named “WUSTL016384_ORD1_ST25.txt”, which is 4,740 bytes in size (as measured in MICROSOFT WINDOWS® EXPLORER), are provided herein and are herein incorporated by reference. This Sequence Listing consists of SEQ ID NOs:1-24.

### BACKGROUND OF THE DISCLOSURE

The present disclosure relates generally to glucose transport inhibitors and methods for identifying the inhibitors. Particularly, inhibitors of glucose transporter Plasmodium falciparum hexose transporter (PfHT) are provided herein. Further, the present disclosure relates to a cell-based, high-throughput assay that directly measures the ability of a compound to inhibit glucose transport by PfHT. This assay determines the intracellular glucose concentration via an expressed glucose sensor protein that changes its fluorescence resonance energy transfer (FRET) intensity in a glucose-dependent manner. This allows for direct assessment of the ability of the compound to inhibit glucose uptake with high accuracy, while eliminating the need for radiolabeled substrates.

Malaria is estimated to have killed half of all the people who have ever lived and remains a major threat, affecting over 200 million people per year. Beyond the staggering effect of this disease on human life, malaria also cripples economic development and burdens the health care systems of malaria endemic countries. The emergence of parasites with resistance to even the most potent existing anti-malarial drugs, such as the artemisinins, has made paramount the development of drugs that target essential pathways for parasite survival. Glucose is the primary source of energy for blood-stage parasites, which almost exclusively rely on glycolysis for ATP production. The malarial glucose transporter, Plasmodium falciparum hexose transporter (PfHT), shown to be essential for parasite survival, is one highly promising molecular target. PfHT has been chemically validated as an antimalarial target.

As a glucose analog, compound 3361, has been found to inhibit PfHT with high selectivity over the human orthologue glucose transporter 1 (GLUT1) and also inhibits asexual intraerythrocytic growth in culture. Compound 3361 is also active against P. berghei liver and transmission stage parasites in infected mice, suggesting that PfHT is a promising target for full life cycle activity. While compound 3361 validates efforts to target PfHT, this compound is not itself considered drug-like, and is therefore not a valid candidate for lead development.

Further, other therapies, such as the HIV protease inhibitor lopinavir, have been identified as targeting PfHT. However, lopinavir has a relatively high IC₅₀ of 16 μM in parasites and shows higher selectivity for the human insulin-responsive glucose transporter GLUT4 over PfHT.

Accordingly, novel therapeutics targeting PfHT with improved potency and selectivity are required.

### BRIEF DESCRIPTION OF THE DISCLOSURE

The present disclosure is generally related to glucose transport inhibitors and methods for identifying the inhibitors. Particularly, inhibitors of glucose transporter Plasmodium falciparum hexose transporter (PfHT) are provided herein. Further, the present disclosure relates to a cell-based, high-throughput assay that directly measures the ability of a compound to inhibit glucose transport by PfHT.

In one aspect, the present disclosure is directed to a compound for inhibiting a glucose transporter. The compound is selected from the group consisting of MMV020548, MMV665941, MMV009085, MMV000326, MMV665879, MMV665898, and combinations thereof.

In another aspect, the present disclosure is directed to an assay for identifying an inhibitor of a glucose transporter. The assay comprises: a cell and an intracellular glucose sensor protein.

In yet another aspect, the present disclosure is directed to a method of screening a compound for inhibition of a glucose transporter. The method comprises: transfecting a cell with a nucleic acid encoding an intracellular glucose sensor protein; contacting the transfected cell with a glucose source; and detecting an intracellular glucose concentration in the transfected cell.

### DETAILED DESCRIPTION

The patent or application file contains at least one drawing executed in color. Copies of this patent or patent application publication with color drawing(s) will be provided by the Office upon request and payment of the necessary fee.

The disclosure will be better understood, and features, aspects and advantages other than those set forth above will become apparent when consideration is given to the following detailed description thereof. Such detailed description makes reference to the following drawings, wherein:

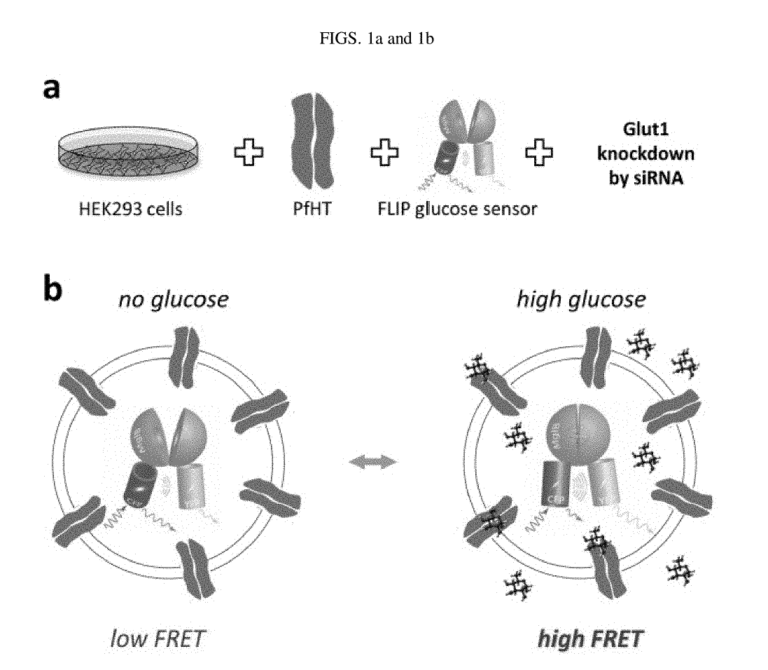

FIG. 1a is a schematic illustrating the generation of cell lines for high throughput screen.

FIG. 1b is a schematic illustrating glucose FRET sensor expressed in PfHT expressing cells.

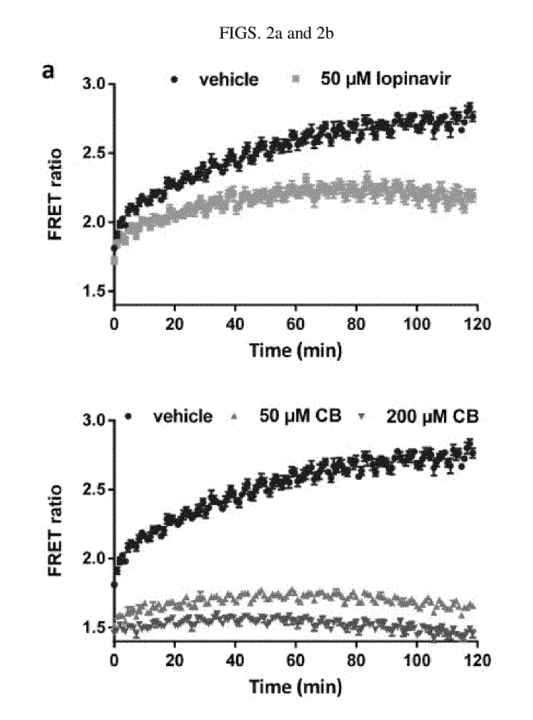

FIG. 2a is a graph depicting the time-dependent change in FRET ratio (YFP emission/CFP emission) after addition of glucose to PfHT-FLIP cells in the presence or absence of lopinavir.

FIG. 2b is a graph depicting the time-dependent change in FRET ratio after addition of glucose to PfHT-FLIP cells in the presence or absence of cytochalasin B (CB).

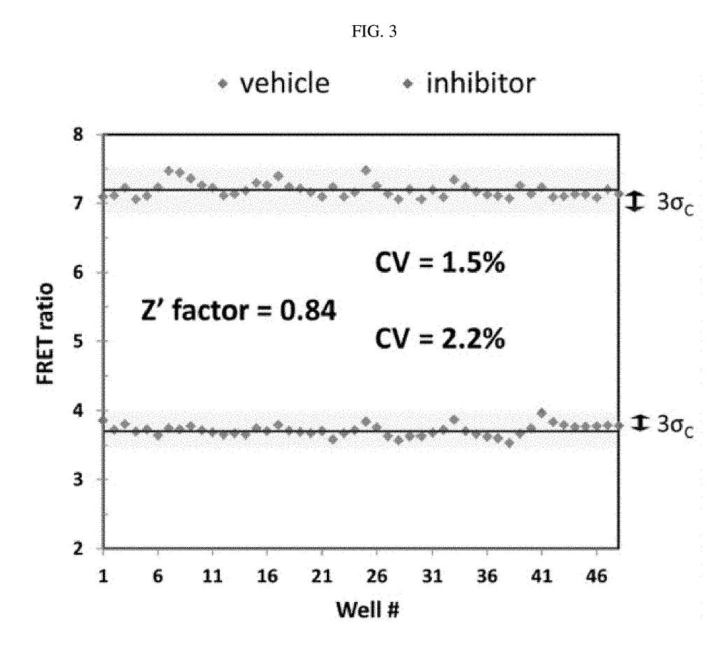

FIG. 3 is a graph depicting FRET ratio per well of 96-well plate, with 48 wells treated with 200 μM CB (inhibitor) and 48 wells treated with vehicle. Z′-factor and the coefficient of variation (CV) were determined from the average and standard deviation of each set of wells.

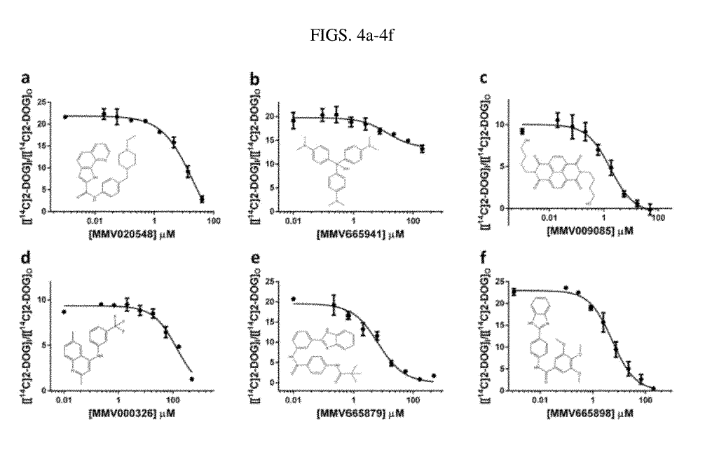

FIGS. 4a-4f are graphs depicting uptake of [¹⁴C]-2-DOG by isolated P. falciparum trophozoites at increasing concentrations of hit compounds. Uptake data are expressed as means±SEM. Chemical structures of compounds are shown for comparison.

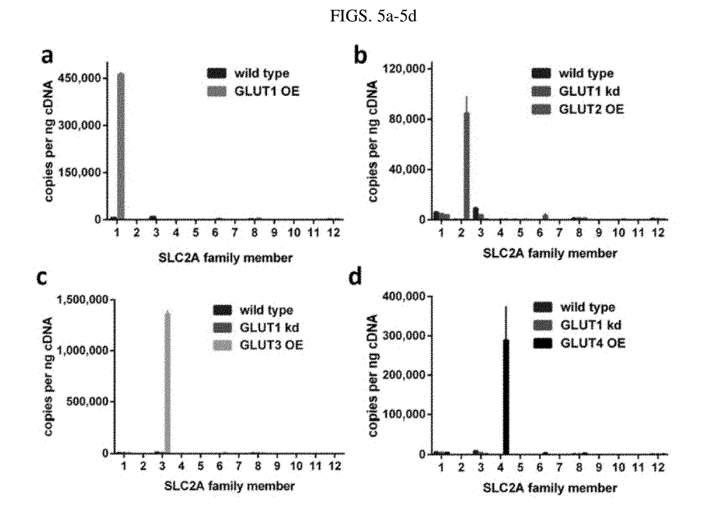

FIGS. 5a-5d are graphs depicting copies of transcript per ng of cDNA for each glucose transporter SLC2A family member in HEK293-FLIP cells overexpressing hGLUT1 (5a), hGLUT2 (5b), hGLUT3 (5c), and hGLUT4 (5d) in comparison to HEK293 wild-type and/or HEK293 wild-type cells treated with siRNA targeting hSLC2A1 (hGLUT1 kd).

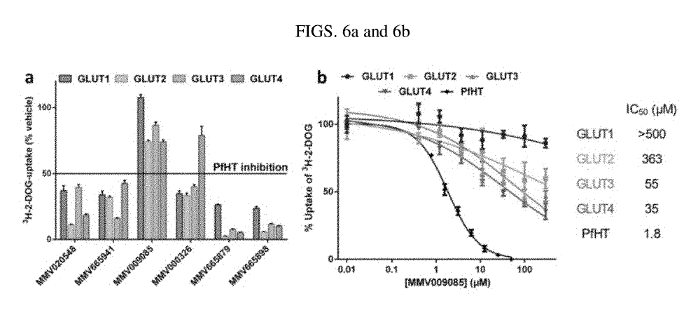

FIGS. 6a and 6b are graphs depicting specificity of hit compounds for PfHT over human orthologues. FIG. 6a depicts uptake of ³H-2-DOG by HEK293 cells overexpressing hGLUTs 1-4 in the presence of hit compounds at their IC₅₀ for PfHT (see, Table 1). FIG. 6b is a graph depicting uptake of [³H]-2-DOG by HEK293 cells overexpressing PfHT or hGLUTs 1-4 at increasing concentrations of compound MMV009085. Uptake data are expressed as means±SEM. IC₅₀ values were calculated using non-linear regression analysis.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

Methods of Identifying Glucose Transporter Inhibitors

To develop a robust and efficient high-throughput assay to identify novel glucose transporter inhibitors, consideration was given to simplicity, sensitivity, scalability, cost, and reliability. Current assays for transporter inhibition in high-throughput format generally employ radiolabeled substrate or cell death of a transporter-overexpressing cell line as a readout. Both formats have significant limitations. Although measuring the uptake of radiolabeled substrate generally yields quantitative, highly reproducible results and minimizes false positives (e.g., fluorescent compounds), the use and disposal of radiolabels are expensive and handling radioactive substances requires increased safety precautions. Alternatively, using cell death of an engineered cell line that requires transporter function for survival is an elegant way to simplify the readout. In both cases, however, these assays fail to discriminate between compounds that kill the cells through transporter inhibition and compounds that kill through other mechanisms, resulting in false positive results as high as 97.8%.

The ideal assay would evaluate transporter function as a direct, highly reproducible readout without the use of radiolabels. Therefore, in the present disclosure, there was designed a cell line that transports glucose almost exclusively through PfHT, which was combined with an intracellular glucose sensor protein that translates glucose concentration into a fluorescence signal as readout.

In one aspect, the human embryonic kidney cell line HEK293 was used. Particularly, a reporter cell line, stably expressing PfHT in conjunction with FLIP (PfHT-FLIP cells), was created. HEK293 cells are known to exhibit relatively low endogenous glucose uptake. To further reduce background levels of glucose uptake, the primary endogenous transporter isoform GLUT1 can be knocked down using siRNA, yielding a cell line that predominantly transports glucose through PfHT. Other suitable cells include COS-7 cells, NSO cells, and CHO-DG44 cells.

Cells can suitably be stably transfected with pfHT DNA, human GLUT1 DNA (Glucose Transporter Type 1), human GLUT2 DNA, human GLUT3 DNA, human GLUT4 DNA, human GLUT5 DNA, human GLUT6 DNA, human GLUT7 DNA, human GLUT8 DNA, human GLUT9 DNA, human GLUT10 DNA, human GLUT11 DNA, human GLUT12 DNA and HMIT. The ability to measure the activity of each of the GLUTs provides an unparalleled ability to test for potential toxicity. The screening assay provides the ability to assess candidate compounds for specific GLUT isoform selectivity over other GLUT isoforms using a rational optimization approach.

Additionally, in one aspect, the glucose sensor protein used in the assay of the present disclosure was FLII12Pglu-700μδ6 (FLIP). This genetically encoded optical glucose sensor consists of three protein domains: a central glucose-binding domain that is coupled thterminally to a cyan fluorescent protein (CFP) and a yellow fluorescent protein (YFP). Upon excitation of CFP, energy is transferred to YFP through FRET. When glucose enters the cell, it binds to the glucose binding domain, leading to a conformational change that brings the two fluorescent proteins closer together and increases FRET (see e.g., FIG. 1). Other FRET pairs for use as FRET biosensors include, for example, small organic dyes, fluorescent proteins, and quantum dots (as disclosed in Bajar et al., Sensors 2016, 16, 1488, which is hereby incorporated by reference in its entirety). Particularly suitable FRET pairs include fluorescent proteins (as provided in Bajar et al., Sensors 2016, 16, 1488).

Glucose Transporter Inhibitors

Using the assay of the present disclosure, glucose transporter inhibitors were identified. The inhibitors are shown in Table 1.

TABLE 1
1: Glucose Transporter Inhibitors

| | | | IC₅₀ |
|---|---|---|---|
| | | IC₅₀ | PfHT |
| | | 3D7 | in |
| | | in | para- |
| Compound | | vitro | sites |
| ID # | Formula | (μM) | (μM) |
| MMV020548 | 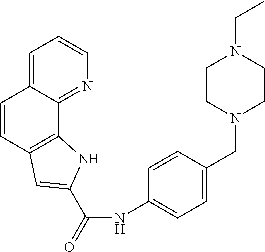 [SMILES: CCN1CCN(Cc2ccc(NC(=O)c3cc4ccc5cccnc5c4[nH]3)cc2)CC1] | 0.114 | 21.3 |
| MMV665941 | 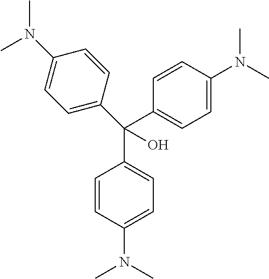 | 0.026 | 13.3 |
| MMV009085 | 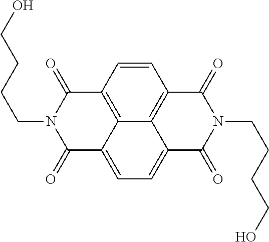 [SMILES: O=C1c2ccc3c4c(ccc(c24)C(=O)N1CCCCO)C(=O)N(CCCCO)C3=O] | 0.987 | 1.8 |
| MMV000326 | 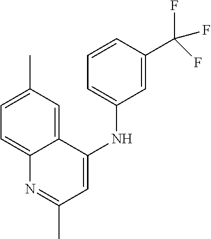 [SMILES: Cc1ccc2nc(C)cc(Nc3cccc(C(F)(F)F)c3)c2c1] | 0.344 | 139 |
| MMV665879 | 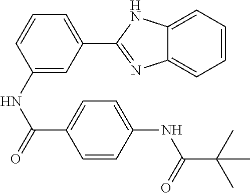 [SMILES: CC(C)(C)C(=O)Nc1ccc(C(=O)Nc2cccc(-c3nc4ccccc4[nH]3)c2)cc1] | 0.390 | 6.6 |
| MMV665898 | 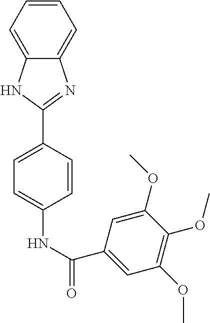 [SMILES: COc1cc(C(=O)Nc2ccc(-c3nc4ccccc4[nH]3)cc2)cc(OC)c1OC] | 0.487 | 6.2 |

### Examples

### Materials and Methods

[¹⁴C]-2-deoxy-glucose (2-DOG) and [3H]-2-DOG were purchased from American Radiolabels Inc. GLUT1 shRNA was obtained through the RNAi core at Washington University School of Medicine. HEK293 cells were acquired from the American Type Culture Collection (ATCC). Lopinavir was obtained through the NIH AIDS Reagent Program, Division of AIDS, NIAID, NIH.

HEK293 cells were transfected with pcDNA3.1 FLII12Pglu-700uDelta6 (Addgene, Cambridge, Mass.), containing the FRET glucose sensor (HEK293-FLIP) using Optifect Reagent (Life Technologies, Waltham, Mass.) according to manufacturer's specifications. Cells that stably integrated the gene were selected using G418 (Sigma Aldrich, St. Louis, Mo.) and highest expressers were identified using Fluorescence Activated Cell Sorting (FACS). These cells were then stably transfected with PfHT, human GLUT1 (hGLUT1), human GLUT2 (hGLUT2), human GLUT3 (hGLUT3) or human GLUT4 (hGLUT4) DNA in the pcDNA 3.1(−) hygro plasmid (Life Technologies). Single clones were selected by comparing their ability to transport radiolabeled glucose. In all cell lines except for HEK293 overexpressing hGLUT1 cells, native hGLUT1 was knocked down using shRNA.

To quantify mRNA transcript levels in the four GLUT overexpressing cell lines, total RNA was isolated using the TrizolW Plus RNA Purification System (Invitrogen, Carlesbad, Calif.), and one microgram of RNA was reverse transcribed using qScript cDNA Supermix (Quanta Biosciences, Beverly, Mass.). Quantitative RT-PCR was performed using Power SYBR Green PCR Master Mix (Applied Biosystems, Foster City, Calif.). Each reaction was run in triplicate using the primers listed in Table 2. Quantifications were performed with standard curves generated using plasmids containing each human GLUT (DNASU).

TABLE 2

| | | | | | Amplic |
|---|---|---|---|---|---|
| | SEQ | | SEQ | | on |
| | ID | | ID | | size |
| | NO | Forward primer: | NO | Reverse primer: | (bp) |
| GLUT1 | 1 | AACTCTTCAGCCAGG | 2 | CACAGTGAAGATGAT | 140 |
| | | GTCCAC | | GAAGAC | |
| GLUT2 | 3 | TTTCAGGCCTGGTTC | 4 | GATGGCCAGCTGATG | 86 |
| | | CTATG | | AAAAG | |
| GLUT3 | 5 | ACTTTGACGGACAAG | 6 | ACCAGTGACAGCCAA | 180 |
| | | GGAAATG | | CAGG | |
| GLUT4 | 7 | CATGCTGGTCAACAA | 8 | CCAATGAGGAATCGT | 105 |
| | | TGTCC | | CCAAG | |
| GLUT5 | 9 | GTCGGCCCCTTGGTG | 10 | AATGATGTGGCGACT | 110 |
| | | AATAA | | CTGCT | |
| GLUT6 | 11 | GGCTGCTCATGTCTG | 12 | GATGGCCGCGAAGAA | 161 |
| | | AGGTC | | GAAGAA | |
| GLUT7 | 13 | TGCAGGCATCTCCTA | 14 | ACGAAAACCTCGGTC | 96 |
| | | CAGC | | ATTGTTC | |
| GLUT8 | 15 | CCATCTTTGAAGAGG | 18 | ATGACCACACCTGAC | 145 |
| | | CCAAG | | AAGACC | |
| GLUT9 | 17 | TGGCAAAGATCCCAT | 20 | AGGTGCTCAATGACC | 85 |
| | | ACGTC | | AAACC | |
| GLUT10 | 19 | GCTGTCCTGCAATCC | 22 | GTCCGGCCTCGCATG | 173 |
| | | CTCAG | | TTATC | |
| GLUT11 | 21 | GGAGTCAATGCAGGT | 24 | CCAGAGCCGTAAAGA | 112 |
| | | GTGAG | | TGGCTG | |
| GLUT12 | 23 | TGCTGGATTAAGCCA | 26 | TGGCTAAGGACAGCC | 83 |
| | | CACTG | | ATTTC | |

Several conditions were found that increased the sensitivity and reliability of the assay. 96-well plates were pretreated with 25 μg/ml Polyethylenimine (PEI, 750 kDa, Sigma Aldrich) solution containing 150 mM NaCl to maintain cell adhesion during washing steps. After 20 min incubation at room temperature (RT), PEI was aspirated and wells were air dried for 5 min. PfHT-FLIP cells were plated 48 h prior to the assay in black opaque 96-well plates (Greiner Bio-One, Monroe, N.C.), which were previously treated with PEI, at 30,000 cells/well. After cell plating, plates were then left in the sterile hood at RT for 45 min before transfer to the incubator to reduce edge effects. Compound screening was performed using the integrated and automated screening system (Beckman Coulter, Brea, Calif.) at the Washington University High Throughput Screening Core. SAMI EX software (Beckman Coulter) was used to design and execute the screening protocol. The Malaria Box (Medicines for Malaria Venture, Geneva, Switzerland) compound library was prediluted in glucose-free HEPES buffered saline solution (HBSS) (146 mM NaCl, 4.7 mM KCl, 0.6 mM MgSO₄, 1.6 mM NaHCO₃, 0.13 mM NaH₂PO₄, 2.5 mM CaCl₂, 20 mM HEPES, pH 7.3) using the BiomekFX liquid handler (Beckman Coulter). A 1:100 dilution of 1 mM stock solution was prepared for 10 μM final concentration.

To initiate the screening assay, cells were washed twice with 150 μl HBSS per well using the ELx405 Plate Washer (Biotek, Winooski, Vt.). Cells were starved in HBSS for 30 min at RT, followed by aspiration of the HBSS from cell plates using the ELx405 Plate Washer. Cells were treated with 45 μl of diluted compound using the BiomekFX (with 3 replicate cell plates for each library plate) and incubated for 6 min at RT. To initiate the uptake, 5 μl of 100 mM glucose was added to the wells (final concentration 10 mM) using a Multidrop384 dispenser (Thermo Fisher Scientific, Waltham, Mass.). After incubation at RT for 120 min, fluorescence was measured using the 2102 EnVision Multilabel Plate Reader (Perkin Elmer, Waltham, Mass.) at excitation 436 nm (CFP) and emissions 485 nm (CFP) and 535 nm (YFP).

P. falciparum strain 3D7 was obtained from the Malaria Research and Reference Reagent Resource Center (MR4). P. falciparum parasites were cultured in a 2% suspension of human erythrocytes and RPMI 1640 (Sigma Aldrich) medium supplemented with 27 mM sodium bicarbonate, 11 mM glucose, 5 mM HEPES, 1 mM sodium pyruvate, 0.37 mM hypoxanthine, 0.01 mM thymidine, 10 μg/ml gentamicin, and 0.5% Albumax (Gibco, Waltham, Mass.) at 37° C., 5% O₂/5% CO₂/90% N₂ atmosphere.

Asynchronous P. falciparum cultures were diluted to 1% parasitemia and were treated with compounds at concentrations ranging from 9.8 nM-20 μM. Growth inhibition assays were performed in 100 μl cultures in opaque 96-well plates. Parasite growth was quantified after 3 days by measuring DNA content using PicoGreen (Life Technologies). Fluorescence was measured by a FLUOstar Omega microplate reader (BMG Labtech, Cary, N.C.) at 485 nm excitation and 528 nm emission. IC₅₀ values were calculated by nonlinear regression analysis using GraphPad Prism software.

P. falciparum strain 3D7 was cultured in 100 mm tissue culture dishes (Techno Plastic Products, Trasadingen Switzerland) in a 2% suspension of human erythrocytes and RPMI 1640 medium until reaching >5% parasitemia. Cells were pelleted via centrifugation and resuspended in RPMI. To determine the uptake of radiolabeled glucose into the parasite, it was isolated from the erythrocyte while removing uninfected erythrocytes. Uptake of [¹⁴C]-2-DOG into isolated parasites was determined at RT using the methods described previously(19). Test compounds were added 5 min prior to the addition of [¹⁴C]-2-DOG (0.2 μCi/ml final concentration). Uptake was quenched after 2 min Data were fit by nonlinear regression analysis using GraphPad Prism software. Uptake of [³H]-2-DOG into HEK293-FLIP cell lines was measured in HEPES-buffered saline at RT for 4 min Data were fit by nonlinear regression analysis using GraphPad Prism software.

In the presence of the HIV protease inhibitor lopinavir (FIG. 2a), previously identified to block PfHT-mediated glucose uptake, PfHT-FLIP cells showed decreased FRET ratio with addition of D-glucose, consistent with inhibition of radiolabeled D-glucose uptake in these cells. Additionally, concentration-dependent inhibition of glucose uptake in PfHT-FLIP cells was confirmed by the glucose transport inhibitor cytochalasin B (CB) as previously shown in Xenopus laevis oocytes. In PfHT-FLIP cells, CB caused a maximal decrease in FRET ratio of 75% at 50 μM consistent with radiolabeled uptake inhibition in oocytes and complete inhibition of D-glucose uptake at 200 μM (FIG. 2b). CB is therefore an ideal positive control for assay optimization and high-throughput screening.

The assay was developed for high-throughput application in a 96-well format using the Z′-factor and the coefficient of variation (CV) as measures of assay robustness. For these measurements, half of the plate was treated with 200 μM CB as positive control and the other half with vehicle prior to the addition of D-glucose. Cells adhered tightly to the plate bottom after treatment of the plate with the highly branched polymer PEI that acted as adhesive, thereby preventing cells from dislodging during washing and buffer exchange steps. Further determination of assay temperature, fluorescence read mode, cell density and cell plating protocol, allowed for routinely obtain a Z′-factor of >0.8 (a perfect assay would have a Z′-factor of 1.0) and CV of about 2% (FIG. 3).

To test the assay conditions in an automated setting for high-throughput application, the MMV Malaria Box, a small library of 400 compounds previously shown to have a cytotoxic effect on malaria parasites was selected. This library contains chemically diverse compounds with half of the compounds having drug-like properties and the other half having been selected as molecular probes. As this compound set was selected for their cytotoxic activity against blood-stage malaria parasites, the molecular drug targets remained to be determined. The library was chosen to identify compounds that inhibited parasite growth mainly or partially through blockade of PfHT-mediated glucose uptake, since deorphanization of these compounds (i.e., identification of the direct molecular targets of anti-antimalarial action) can aid in further target directed drug development.

The Malaria Box was screened at 10 μM drug concentrations in triplicate and selected hits that decreased the FRET ratio by more than 30% with vehicle-treated cells set to 100% and cells treated with 200 μM CB set to 0%. Since fluorescent compounds have the potential to generate false positive hits, the FRET ratio of compounds without cells present was determined. After false positive elimination, 5 compounds were identified as primary hits. Hit confirmation was ascertained by determining the IC₅₀ for glucose uptake into isolated P. falciparum parasites from blood culture using radiolabeled D-glucose. Four of the five primary hits were confirmed to inhibit glucose uptake into P. falciparum parasites in the low micromolar range (Table 3) with compound MMV665941 showing only partial inhibition at the highest concentration tested (FIG. 4). Compound MMV020548 was included for comparison as it was previously identified from the same library. The similar IC₅₀ observed in the freed parasite glucose uptake assay and in vitro growth inhibition assay for MMV009085, was consistent with the interpretation that this compound is acting through PfHT blockage to inhibit parasite growth (Table 3) although, alternative targets in the parasite might be inhibited as well. Since the measurement of 2-DOG uptake involves both the transport of this sugar and phosphorylation to 2-DOG-6-P, the possibility that the identified compounds also inhibit the malarial hexokinase was not fully excluded. However, the observation that they inhibited 2-DOG uptake with different potency and that compound MMV009085 showed a widely different IC₅₀ when measured in HEK293 cells overexpressing different GLUTs (FIG. 6) suggested that hexokinase was not directly targeted.

TABLE 3
Comparison of hit IC₅₀ values for different assays.^{a}

| | Heterologous | | EC₅₀ for | EC₅₀ for | |
|---|---|---|---|---|---|
| | screening | IC₅₀ for | P. falciparum | P. falciparum | IC₅₀ PfHT |
| | assay | glucose uptake | growth | growth | (determined by |
| Compound | (% inhibition | in freed | inhibition | inhibition | Ortiz et al. (15) in |
| ID # | at 10 μM) | parasites | 3D7 Strain | Malaria box | L. mexicana) |
| MMV020548 | 21 | 15.5 | 0.114 | 0.361 | 3.5 |
| MMV665941 | 31 | 19.6 | 0.026 | 0.255 | ND |
| MMV009085 | 87 | 2.6 | 0.987 | 0.795 | 0.051 |
| MMV000326 | 49 | 286 | 0.344 | 1.170 | ND |
| MMV665879 | 49 | 8.3 | 0.390 | ND | 2.7 |
| MMV665898 | 64 | 5.3 | 0.487 | 1.220 | ND |

^{a}IC₅₀s for PfHT in P. falciparum were determined from dose-response curves shown in FIG. 4. IC₅₀ values for inhibition of growth of P. falciparum strain 3D7 intraerythrocytic forms by each compound were determined via a growth inhibition assays as described in Material and Methods. EC₅₀ values provided with the malaria box and IC₅₀ values determined for inhibition of glucose uptake into PfHT overexpressing L. mexicana parasites were also tabulated.
ND indicates not determined.

To establish selectivity of these hits for PfHT over human orthologues, the inhibition of GLUTs 1, 2, 3 and 4 was cross-validated by the confirmed hits. Using the same HEK293-FLIP cell line, each of the individual class I transporters was overexpressed. With the exception of GLUT1-FLIP, the other GLUT cell lines were also treated with GLUT1 specific shRNA to reduce background glucose uptake. That the overexpressed GLUT was the main glucose transporter expressed in each cell line was confirmed by comparing the cDNA copy number of all human SLC2A genes via qPCR (FIG. 5). Total transcript number differed between various GLUT isoform overexpressing cell lines as the construct was integrated randomly into the genome of the host cell as discussed in the Material and Methods section. The inhibitory effect of the five confirmed hits was determined on all four human class I GLUTs at the IC₅₀ concentration for PfHT-mediated glucose uptake inhibition. Only compound MMV009085, the most potent hit with an IC₅₀ of 2.6 μM for PfHT-mediated glucose uptake, showed significantly less inhibition of the human GLUTs compared to PfHT (FIG. 6a). Comparison of the IC₅₀s for glucose uptake inhibition, mediated by either the human GLUTs or PfHT, revealed a more than 10-fold higher selectivity of MMV009085 for PfHT over human orthologues (FIG. 6b). Although MMV009085 is considered a probe-like molecule, its high potency in inhibiting both glucose uptake and growth of the parasites as well as its high selectivity for PfHT over human orthologues makes this compound a potential candidate for lead optimization. Additionally, it demonstrated that the newly developed assay can identify glucose transporter-specific inhibitors and further distinguish compounds with high selectivity for their target over its orthologues or isoforms.

The results presented herein demonstrate the development of a novel assay system adaptable to high-throughput screening for small molecules that inhibit glucose uptake. The ability to identify compounds with selectivity for the malarial glucose transporter PfHT over human GLUT transporters provides a powerful new method to identify safe and effective anti-malarial agents. The results showed high reproducibility with a CV of about 2% and good separation of hits from background with a Z′-factor of >0.8, indicative of a high quality assay for high-throughput screening. Additionally, by not requiring radioactive labels, this assay increases throughput and ease-of-use with substantially reduced cost. This is especially important in the field of infectious diseases affecting people in the developing world. The assay employs cells that genetically encode the glucose transporter and a glucose sensor utilizing a direct fluorescent readout. This obviated the need for time consuming protein purification steps and expensive radiolabeled or fluorescently labeled substrates. Every step in the assay was successfully tested in a fully automated setup in order to test the true scalability to high-throughput. The initial screen of a 400 compound library resulted in four verified hits. Although the rate of false positive compounds identified after the initial round of screening was significantly lower than in a previously reported PfHT inhibitor assay, further improvements can be made by selecting a higher threshold of FRET ratio reduction, especially when screening a larger library or using different detection methods like fluorescence lifetime. The hits identified in this screening assay included three compounds (MMV009085, MMV020548, MMV665879) that were previously identified and characterized to selectively inhibit glucose uptake, but not proline transport, in PfHT-overexpressing Leishmania mexicana. However, the L. mexicana assay system showed non-correlated results for the IC₅₀ of compound MMV009085 (malaria parasite growth assay=0.99 μM, yet L. mexicana PfHT overexpression assay=0.051 μM). This 20-fold difference in potency by the two assays indicates that in the cross-expression system (PfHT in the Leishmania parasite) MMV009085 might have an alternative target, originating from L. mexicana. In the screening assay of the present disclosure, reduction in FRET ratio correlated with inhibitory potency of the compound in the freed parasite glucose uptake assay (R²=0.83, excluding outlier MMV000326) with the most potent inhibitor MMV009085 showing the strongest decrease in FRET ratio.

The quality of hits in drug discovery projects is crucial in improving the odds of developing a successful candidate for clinical application, especially as downstream investments into the hits that are selected for lead development are significant both in terms of funding and time. Therefore, target-based screens have advantages to phenotypic screens, as they allow lead identification and optimization that are directed towards the malarial target protein versus human orthologues. The assay system of the present disclosure was adapted to include counter-screening of PfHT-specific hits against human glucose transporters. The facilitative glucose transporters GLUT1-4 are fundamentally important for human glucose homeostasis as they are primary glucose transporters in most tissues. PfHT and the human transporters share a common MFS fold and show a sequence similarity of close to 50%. The significance of the human GLUTs and their close homology to the malarial glucose transporter make it crucial, but potentially challenging, to identify drugs with high parasite orthologue selectivity. By replacing PfHT with human GLUTs, the HEK293 cell based assay system successfully distinguished PfHT-specific from orthologue non-specific inhibitors and identified a compound that showed a 19 to >250-fold high selectivity for PfHT over the human GLUTs.

An additional advantage of target-directed screening is the opportunity for structure-based rational drug design to aid in lead compound optimization, particularly with respect to selectivity for PfHT over human GLUTs. For the mammalian glucose transporters, recent progress has been made in solving the crystal structures of several isoforms in both inward and outward conformations. Furthermore, prior work provided evidence for the binding pocket that mediates transporter inhibition by HIV protease inhibitors. This has been used to model lopinavir binding to a model of PfHT. Taken together, these data provide a framework for iterative structure-activity analysis of compounds identified via the high-throughput screening assay of the present disclosure.

In addition to using the GLUT-expressing FLIP cells lines for selecting orthologue-specific PfHT inhibitors, these cell lines can be used to identify GLUT-specific inhibitors with isoform selectivity with applications beyond anti-malarial therapy. Several types of tumors have been shown to overexpress specific GLUT (glucose transporter) isoforms that mediate the transport of high levels of glucose to malignant cells that have undergone metabolic rearrangement and primarily metabolize glucose through aerobic glycolysis (known as the Warburg effect). Many cancer cells express transporter isoforms that are not found in these tissues in non-malignant conditions. Among the class 1 facilitative glucose transporters, GLUT1 is most abundantly expressed in cancers from breast, brain, lung, colorectal, and bladder. Inhibition of GLUT1 has already been shown to exert antineoplastic effects, both in vitro and in vivo making it a target for cancer therapy. Expression of the glucose transporters GLUT2, GLUT3 and GLUT4 is also upregulated in various tumors, among them several types that are difficult to treat and are associated with low survival rates like lung, pancreatic, and liver tumors. The cell-based, target-specific screen of the present disclosure is adaptable to mediate glucose transport through selectively expressed transporter isoforms. This versatility combined with a robust, easily detectable readout significantly increases the feasibility of screening large compound libraries to identify isoform-specific GLUT inhibitors that can be further developed into drugs for either stand-alone or adjunct cancer therapy. cm What is claimed is:

1. A compound for inhibiting a glucose transporter, the compound being selected from the group consisting of MMV020548, MMV665941, MMV009085, MMV000326, MMV665879, MMV665898, and combinations thereof.

2. The compound of claim 1, wherein the compound is MMV009085.

3. The compound of claim 1, wherein the glucose transporter is Plasmodium falciparum hexose transporter (PfHT), human GLUT1 DNA, human GLUT2 DNA, human GLUT3 DNA, human GLUT4 DNA, human GLUTS DNA, human GLUT6 DNA, human GLUT7 DNA, human GLUT8 DNA, human GLUTS DNA, human GLUT10 DNA, human GLUT11 DNA, human GLUT12 DNA and HAUT.

4. An assay for identifying an inhibitor of a glucose transporter, the assay comprising: a cell and an intracellular glucose sensor protein.

5. The assay of claim 4, wherein the cell is a human embryonic kidney cell, a COS-7 cell, a NSO cell, and a CHO-DG44 cell.

6. The assay of claim 4, wherein the glucose sensor protein is FLII12Pglu-700μδ6 (FLIP).

7. A method of screening a compound for inhibition of a glucose transporter, the method comprising:
transfecting a cell with a nucleic acid encoding an intracellular glucose sensor protein;
contacting the transfected cell with a glucose source; and
detecting an intracellular glucose concentration in the transfected cell.

8. The method of claim 7, wherein the cell is a human embryonic kidney cell.

9. The method of claim 7, wherein the glucose sensor protein is FLII12Pglu-700μδ6 (FLIP).

10. The method of claim 7, wherein the intracellular glucose concentration is detected using fluorescence resonance energy transfer (FRET).


SEQUENCE LISTING (24 sequences)

SEQ ID NO: 1 - DNA, 21 bp, Artificial Sequence
  Synthetic
aactcttcagccagggtccac

SEQ ID NO: 2 - DNA, 21 bp, Artificial Sequence
  Synthetic
cacagtgaagatgatgaagac

SEQ ID NO: 3 - DNA, 20 bp, Artificial Sequence
  Synthetic
tttcaggcctggttcctatg

SEQ ID NO: 4 - DNA, 20 bp, Artificial Sequence
  Synthetic
gatggccagctgatgaaaag

SEQ ID NO: 5 - DNA, 22 bp, Artificial Sequence
  Synthetic
actttgacggacaagggaaatg

SEQ ID NO: 6 - DNA, 19 bp, Artificial Sequence
  Synthetic
accagtgacagccaacagg

SEQ ID NO: 7 - DNA, 20 bp, Artificial Sequence
  Synthetic
catgctggtcaacaatgtcc

SEQ ID NO: 8 - DNA, 20 bp, Artificial Sequence
  Synthetic
ccaatgaggaatcgtccaag

SEQ ID NO: 9 - DNA, 20 bp, Artificial Sequence
  Synthetic
gtcggccccttggtgaataa

SEQ ID NO: 10 - DNA, 20 bp, Artificial Sequence
  Synthetic
aatgatgtggcgactctgct

SEQ ID NO: 11 - DNA, 20 bp, Artificial Sequence
  Synthetic
ggctgctcatgtctgaggtc

SEQ ID NO: 12 - DNA, 21 bp, Artificial Sequence
  Synthetic
gatggccgcgaagaagaagaa

SEQ ID NO: 13 - DNA, 19 bp, Artificial Sequence
  Synthetic
tgcaggcatctcctacagc

SEQ ID NO: 14 - DNA, 22 bp, Artificial Sequence
  Synthetic
acgaaaacctcggtcattgttc

SEQ ID NO: 15 - DNA, 20 bp, Artificial Sequence
  Synthetic
ccatctttgaagaggccaag

SEQ ID NO: 16 - DNA, 21 bp, Artificial Sequence
  Synthetic
atgaccacacctgacaagacc

SEQ ID NO: 17 - DNA, 20 bp, Artificial Sequence
  Synthetic
tggcaaagatcccatacgtc

SEQ ID NO: 18 - DNA, 20 bp, Artificial Sequence
  Synthetic
aggtgctcaatgaccaaacc

SEQ ID NO: 19 - DNA, 20 bp, Artificial Sequence
  Synthetic
gctgtcctgcaatccctcag

SEQ ID NO: 20 - DNA, 20 bp, Artificial Sequence
  Synthetic
gtccggcctcgcatgttatc

SEQ ID NO: 21 - DNA, 20 bp, Artificial Sequence
  Synthetic
ggagtcaatgcaggtgtgag

SEQ ID NO: 22 - DNA, 21 bp, Artificial Sequence
  Synthetic
ccagagccgtaaagatggctg

SEQ ID NO: 23 - DNA, 20 bp, Artificial Sequence
  Synthetic
tgctggattaagccacactg

SEQ ID NO: 24 - DNA, 20 bp, Artificial Sequence
  Synthetic
tggctaaggacagccatttc